(19) United States
(12) Patent Application Publication
(10) Pub. No.: US 2010/0129466 A1
(43) Pub. Date: May 27, 2010

(54) COMPOSITIONS AND METHODS FOR TREATING PLANTS

(75) Inventors: Lyriam L. Marques, Edmonton (CA); Merle E. Olson, Calgary (CA); Michael W. Harding, Rosemary (CA)

Correspondence Address: WILLIAM J BUNDREN;THE LAW OFFICE OF WILLIAM J BUNDREN, 734 LaRue Road, Millersville, MD, 21108, US

(73) Assignee: INOVOTECH, INC., Edmonton, AB (CA)

(21) Appl. No.: 11/913,158

(22) PCT Filed: Jun. 22, 2007
(86) PCT No.: PCT/CA2007/001149

§ 371 Date: Feb. 12, 2010

Related U.S. Application Data
(60) Provisional application No. 60/815,723, filed on Jun. 22, 2006.

(51) Int. Cl. A01N 59/16; C22C 5/06

(52) U.S. Cl. 424/618; 420/501

(57) ABSTRACT

The invention is a method and composition for treating biofilm using a high valency silver ion. In preferred embodiments of the invention, the anti-biofilm agent is used to preserve, disinfect or treat plant material, including seeds, leaves, stems, vessels, flowers, roots and fruits, and any surface, particularly disinfecting work or processing surfaces and seed or plant surfaces; in anti-microbial coatings; and in treating human, plant, and animal diseases and conditions

### I. FIELD OF INVENTION

This invention relates to compositions and methods for treating biofilm. The compositions and methods are for preserving plant material or any portion of a plant; and/or for treating, preventing or reducing microbial contamination of plant material. The compositions and methods are also suitable for treating or preventing microbial contamination on any surface that may come into contact with the plant material (i.e. surfaces used for production, handling, transport, storage, processing or packaging). The compositions and methods comprise at least one high valency silver ion.

### II. BACKGROUND OF THE INVENTION

Environmental, medical and industrial microbiologists have documented that microbial populations in their natural environments do not routinely grow as solitary or planktonic cells, but rather as biofilms; complex communities, attached to surfaces (Costerton et al., 1995; Davey and O'Toole, 2003). These discoveries have shifted the conceptual framework for treating a wide variety of microbiological diseases and conditions, including but not limited to plant pathology (Marques et al., 2002; Dow et al., 2002; Ramey et al., 2004); a wide variety of agricultural and farming applications; the food industry, particularly food processing surfaces; food borne illnesses, particularly Salmonella; food contamination and/or disease, including but not limited to Pierce's Disease in grapes, potato ring rot and storage rots, browning root rot, seed infestations; milk and milk products, a wide array of human and animal infections; medical implants; and medical devices.

Plant diseases cause world-wide economic losses in all industries involving agricultural plant production including food commodity production, horticulture, floriculture, nutraceuticals, turf-grass, forages, nursery crops, forestry operations fiber crop production and alternative fuels. In addition, pathogens attack plant materials in post-harvest storages. Global economic losses due to plant diseases were estimated at 10%-15% reduction in potential production resulting in a cost of $76.1 billion between 1988 and 1990 (Orke et al., 1994; Pinstrup-Anderson, 2001). These infections in plants and produce are caused predominantly by microorganisms such as fungi, bacteria, nematodes, protists and viruses.

Conventional commercial washing and sanitizing methods to remove microbial contaminants have been found to be marginally effective when biofilm is involved.

Another major concern in plant production is the occurrence of soil- and seed-borne diseases. As an example, bacterial and fungal pathogens can cause disease and loss to every sector of agriculture. Fungal pathogens such as Pythium spp., Phytophthora spp., Rhizoctonia solani, Fusarium spp., can cause damping off, seed rot, seedling blight and foot rot in a wide array of plants. Bacterial pathogens are a major problem in many crops including the production of dry bean (Phaseolus vulgaris) world-wide (Hirano and Upper, 1983; Singh and Munoz, 1999). Pathogens such as Pseudomonas syringae pv. syringae (brown spot), P. syringae pv. phaseolicola (halo blight), Xanthomonas axonopodis pv. phaseoli (common blight) and Curtobacterium flaccumfaciens pv. flaccumfaciens (wilt) cause serious losses in bean fields if the diseases are not managed. The use of certified disease-free seed is the first line of defense in preventing infections. Once diseases are introduced, the only method of control is the application of registered chemical pesticides. Foliar pesticides can reduce disease pressure; however, chemical treatments applied to diseased fields must be applied repeatedly from the onset of symptoms until near harvest.

A complicating factor in seed pathology is the ability of pathogenic bacteria to form biofilms, which are often highly resistant to removal and disinfection (Costerton et al. 1999; Ceri et al. 2001; Olson et al. 2002). As a result, past and current experimental results may dramatically overestimate the efficacy of chemicals used as antimicrobial cleaners, pesticides or disinfectants. It has been demonstrated that Fusarium spp. and the four bacterial pathogens of dry bean listed above can and do form biofilms either in vitro or in seeds (unpublished). In addition, these bacteria can form biofilms on seeds, resulting in current seed treatments being ineffective or marginally effective.

There is still a need for an effective anti-biofilm agent with the following properties: inexpensive (or cost-effective), broad-spectrum efficacy, sustained release of anti-biofilm agent, ability to remove or degrade biofilms, and a low level of toxicity. This would be extremely beneficial to a very perishable commodity by lowering costs of disease management, increasing quality and economic value of plant material, increasing customer satisfaction, increasing consumer confidence and promoting industry growth in the agricultural field as well as in the medical and industrial fields. Likewise, more effective seed treatments could help producers in three areas: 1. prevention of seed-borne plant diseases in greenhouse and field crops that would subsequently reduce production losses and costly foliar pesticides applications. 2. Prevention of soil-borne diseases and frost damage (in short-season regions) when seed treatments increase the speed of germination and emergence. 3. Safer pesticides will reduce ecological damage to natural and agricultural environments. Seed treatments can also help reduce the risk of food-borne human infections such as those associated to sprouts.

It is known in the art to employ methods and compositions comprising silver as an anti-microbial agent. The prior art, however, teaches use of silver as an anti-microbial agent against solitary or planktonic cells and not as an anti-biofilm agent against microorganisms growing as biofilms. It is known that covering a growing plant with silver nitrate provides an anti-microbial effect, which helps protect the plant from disease. The traditional understanding, however, is that although a silver treatment could protect seeds from disease, such treatment may not work in practice because it may be deleterious to seed germination or seedling development due to interference with plant hormones/signalling. Also, the prior art teaches using monovalent silver as an anti-microbial agent but does not teach using silver of any higher valency.

There does not exist in the prior art, methods and composition comprising high valency silver ions for use as an antimicrobial and/or anti-biofilm agent to treat, prevent or reduce microbial contamination of seeds, including, but not limited to, microorganisms growing as biofilms on seeds, wherein such methods and compositions do not inhibit seed germination. There is also a need for such methods and compositions to help increase the germination rate and the germination speed of seeds, and to improve avoidance of soil-borne diseases and frost-damage.

### III. SUMMARY OF THE INVENTION

There is a need for methods and compositions for preserving plant material and/or for treating, preventing or reducing microbial contamination of plant material, including but not limited to preserving seeds and/or to treating, preventing or reducing microorganisms growing as biofilms on plant material.

The compositions and methods of the present invention comprise high valency silver ions as the anti-biofilm agent.

The compositions and methods of the present invention have applicability in a wide variety of agricultural, industrial, and medical environments, e.g., extending or improving the life of plant material, disinfecting any surface, particularly disinfecting work or processing surfaces (e.g., tables) and seed or plant surfaces; in anti-microbial coatings; and in treating human, plant, and animal diseases and conditions.

### IV. DETAILED DESCRIPTION OF THE INVENTION

The present invention comprises compositions and methods for treating a biofilm using an anti-biofilm agent comprising silver ions, preferably high valency silver ions. The compositions and methods may also include one or more other active agents. The compositions and methods are anti-microbial, e.g. against biofilm, similar structures, or precursors formed by bacteria, fungi, viruses, algae, or parasites, yeast and other microbes. As described in more detail below, the methods and compositions of the present invention may be used wherever biofilm or similar structures may be found, including but not limited to microorganisms growing and/or floating in liquid environments.

The method comprises treating, preventing or reducing microbial contamination of a seed by contacting said seed with an antimicrobial agent comprising at least one form of high valency silver. The composition comprises at least one form of high valency silver. The method and the composition may be used for treating a seed against planktonic microorganisms.

In some embodiments of the invention, the compositions and methods may be used to treat or prevent one or more biofilms. In some embodiments of the invention, the compositions and methods may be used to treat and/or prevent one or more human, animal, or plant diseases, conditions, infections, or contamination. Typically these diseases and infections, etc., are caused by microbes associated with or residing in the biofilm.

The present invention also comprises compositions and methods to treat, prevent or reduce one or more biofilms growing on plant material, using at least one form of high valency silver, such as for example but not limited to silver ions having Ag (II) and Ag (III) valent states. In one embodiment, the method comprises treating, preventing or reducing biofilm(s) on plant material by contacting the plant material with an anti-biofilm agent comprising at least one form of a high valency silver. In one embodiment, the composition may comprise an anti-biofilm agent comprising at least one form of a high valency silver.

In some embodiments, the present invention comprises compositions and methods for preserving the health, life, or quality of plant material, including treating against bacteria, fungi, algae, biofilms, viruses, and parasites by contacting the plant material with a composition comprising one or more anti-biofilm agents. The anti-biofilm agent comprises one or more high valency silver ions. In some embodiments of the invention, the compositions and methods may be used to preserve and/or disinfect plants, plant material, or parts thereof, most preferably seeds. In some embodiments of the invention, the compositions and methods may be used to extend storage life or preserve the plant material. In some embodiments of the invention, the anti-biofilm agent reduces or eliminates surface contamination.

The compositions and methods may also include one or more other active agents and/or additives. The compositions and methods may further comprise contacting said seed with one or more additional anti-biofilm agents, preservatives and/or additional antimicrobial agents, each of which may comprise at least one form of high valency silver or comprise some other active agent or combinations.

The present invention includes any method of contacting with an anti-biofilm agent. Typical mechanisms of contacting include but are not limited to coating, spraying, immersing, wiping, and diffusing in liquid, powder or other delivery forms (e.g., injection, tablets, washing vacuum or oral). In some embodiments of the invention, the compositions and methods may include applying the anti-biofilm agent to any portion of a plant or plant material. Further, any structure or hard surface (e.g., tools or machinery surfaces associated with harvesting, transport, handling, packaging or processing) can be sanitized, disinfected, impregnated, or coated with the anti-biofilm agent of the present invention.

Further, any storage, or greenhouse facilities or transport container can be impregnated with an anti-biofilm agent of the present invention so that the anti-biofilm agent prevents surface contamination and comes into contact with a plant or a portion thereof.

The compositions of the present invention may be used to treat a plant or portion thereof to eliminate or reduce one or more undesirable and/or deleterious microorganisms. The compositions of the present invention may be used to prevent one or more undesirable or deleterious microorganism from infecting a plant or portion thereof. In these embodiments of the invention, the preservative compositions and methods may be an anti-microbial agent.

The compositions of the present invention may be used to treat a plant or portion thereof to eliminate or reduce one or more undesirable and/or deleterious biofilms. The compositions of the present invention may be used to prevent one or more undesirable or deleterious biofilms from infecting a plant or portion thereof. In these embodiments of the invention, the preservative compositions and methods may be an anti-biofilm agent.

This invention demonstrates that stable, slow release silver ion compounds, can be used as antimicrobials against bacterial and fungal pathogens, including biofilms, growing on plant surfaces or more broadly any hard surfaces associated with bacterial and fungal contaminants, e.g., wood, concrete, metal, rubber or plastic, dental implants, and catheters.

In accordance with some embodiments of the invention, any method of contacting the seed with an antimicrobial and/or anti-biofilm agent may be used. Typical mechanisms for contacting the seed include but are not limited to coating, spraying, immersing, and diffusing in liquid, gel, powder or other delivery forms.

Some embodiments of the invention comprise a composition of the present invention, and its use for preserving and extending the shelf life of a plant or plant material.

Compositions of the present invention include any silver containing compound that produces a high valency silver species, typically formed by the combination of a silver compound and Ag oxide(s). These compositions exhibit antimicrobial activity and/or anti-biofilm activity against a variety of microbes, including both bacteria and fungi, and provide a sustained release of silver ions from silver compounds. The term “oxidized silver species” as used herein may involve but is not limited to compounds of silver where said silver is in +I, +II or +III valent states or any combinations thereof. These oxidized silver species include, for example silver (I) oxide, silver (II) oxide, silver (III) oxide or mixtures thereof, all silver salts having a solubility product higher than 10-20 (such as for example Ag₂SO₄, AgCl, Ag₂S₂O₈, Ag₂SO₃, Ag₂S₂O₃, Ag₃PO₄, and the like), and silver oxy-salts such as Ag₇O₈X were X can include but is not limited at NO³⁻, ClO⁴⁻, SO₄²⁻, F— etc. These active silver species may include but are not limited to oxidized silver species such as silver salts, silver oxide (Ag₂O), higher silver oxides i.e. Ag(II) and Ag(III) (AgO, Ag₂O₃, Ag₃O₄ or like), silver oxy-salts with a general formula Ag₇O₈X where X can include one of acid anions such as sulfates, chlorides, phosphates, carbonates, citrates, tartrates, oxalates and like. The composition may also include elemental silver, preferably in small amounts, as a by-product of the oxidation of production process.

The preferred composition of the present invention comprises an aqueous suspension of any form of silver that results in a high valency silver species. These active silver species may include at least one form of a high valency silver comprising an at least one form of soluble silver ion selected from the group consisting of Ag+, Ag++ and Ag+++.

Silver ions, as used herein, refers to a composition containing silver ions having valent states higher than one, such as for example Ag (II) and Ag (III) valent states. The preferred composition is an aqueous suspension. The compositions and methods of the invention may be comprised of silver ions having more than one valent state so that the oxidized silver species may be comprised of a multivalent substance. Finally, it is believed that the compositions of the present invention may be comprised of a silver-containing substance or a plurality of silver containing substances that react over time to form other silver containing substances which may exhibit differing antimicrobial properties.

In preferred embodiments of the invention, antimicrobial properties may be achieved by contacting an antimicrobially active silver species or high valency silver ion within or at the surface of a substrate, such as a plant material. High valency silver ions may be produced by any process or reaction that produces high valency silver ions. The preferred processes are those that result in an aqueous suspension of high valency silver ions. These processes are well known to those of ordinary skill in the art. See for example, J. A. MacMillan, Chem. Rev., 62, 65 (1962); and S. S. Djokic, J. Electrochemical. Soc. 151, (6) C359 (2004)

In the preferred embodiments, the high valency silver ions may be produced by first providing an aqueous solution of monovalent silver salt or a silver complex such as silver nitrate, perchlorate or silver diamino complex. Silver nitrate is more preferable if the reaction is carried out under acidic conditions or at close to neutral conditions (i.e. at pH below 7). Silver diamino complex, (i.e., [Ag(NH₃)²]⁺) is more preferable if the reaction is carried out under alkaline conditions (i.e. at pH above 7). In preferred embodiments, the oxidizing agent is potassium persulfate (KPS).

Where the methods or compositions comprise at least one silver compound releasing Ag++, the compound may be selected from the group consisting of, but not limited to silver (II) oxide (AgO), high valency silver salts (Ag(Ag₃O₄)X where X=NO₃, ClO₄, F or HSO₄, (Ag₃O₄)₂SO₄, silver(II) sulfate (AgSO₄), silver bifluoride (AgF₂), Silver(II) periodate; organic complexes such as, but not limited to, (Ag py₄S₂O₈, silver ortho-phenanthroline, Agdipy₂, Agdipy₂(X)₂, where X=NO₃, ClO₄, Ag dipy₃, Agdipy₃(X)₂, where X=NO₃, ClO₄, Ag dipy₂(NO₃)₂.NO₃.HNO₃, (AgtripyNO₃)NO₃, silver(II) quinolate, silver(II) cinchomeronate, silver(II) isocinchomeronate, silver(II) lutidinate, silver(II) dipicolinate, silver(II) niconate, silver(II) isoniconate, silver(II) pyridine-2,4,6-3 carboxalate (black), silver(II) pyridine-2,4,6-3 carboxalate (brown), silver(II) pyridine-2,4,5-3 carboxalate, silver(II) biguanide, silver(II) benzalkonium chloride, silver(II) cetyldimethylethylammonium bromide, silver(II) ethylene biguanide.

Where the methods or composition comprise at least one silver compound releasing Ag+++, the compound may be selected from the group consisting of, but not limited to silver(III) fluorides [(BaAgF₅, MAgF₄ (M=K, Rb, Cs, N)], silver (III) periodate [Na₅H₂Ag(III)(IO₆)₂.H₂O], silver(III) tellurate, silver(III) ethylenebis (biguanide) [Ag(enbigH)₂X where X=SO₄, NO₃, ClO₄ or OH], silver(III) biguanide.

In other embodiments, the method or the composition may comprise silver (I, II, III) peroxide, colloidal silver, nanocrystalline silver or silver zeolite.

Methods of producing high valency silver ions are well known to those skilled in the art.

The silver deposition compounds may be used in any of the following formats: silver deposition coatings, liquid, powder, capsule, tablet, coating, and similar configurations. In a preferred embodiment of the present invention, active agents are incorporated directly, or may be incorporated by sequentially adding components or precursors of the active agent to the plant material, and having the precursors of the active agent in or on the coating. Other forms also include films, sheets, fibers, sprays and gels.

The preservative agents incorporated into the composition may be used for a variety of applications where there is a need for the presence of a preservative agent. A preferred use is in the treatment and preservation of plant material in both the agricultural sector, including but not limited to edible and fiber crops, produce, ornamental, nursery plants, tree seedlings, fiber plants, turf grass and forages, oilseeds, cereals, pulses, vegetables, medicinal plants, nutraceutical plants, and greenhouse crops.

The composition may also include additional antimicrobial agents, including but not limited to antifungal agents, antibacterial agents, anti-viral agents and anti-parasitic agents, growth factors, angiogenic factors, anaesthetics, mucopolysaccharides, and metals, disinfectants, antibiotics, cleaners, and other chemicals.

Examples of antimicrobial agents that may be used in the present invention include, but are not limited to: 8-hydroxyquinoline sulfate, 8-hydroxyquinoline citrate, aluminum sulfate, quaternary ammonium, isoniazid, ethambutol, pyrazinamide, streptomycin, clofazimine, rifabutin, fluoroquinolones, ofloxacin, sparfloxacin, rifampin, azithromycin, clarithromycin, dapsone, tetracycline, erythromycin, ciprofloxacin, doxycycline, ampicillin, amphotericin B, ketoconazole, fluconazole, pyrimethamine, sulfadiazine, clindamycin, lincomycin, pentamidine, atovaquone, paromomycin, diclazaril, acyclovir, trifluorouridine, foscarnet, penicillin, gentamicin, ganciclovir, iatroconazole, miconazole, Zn-pyrithione, heavy metals including, but not limited to, gold, platinum, silver, zinc and copper, and their combined forms including, salts, such as chloride, bromide, iodide and periodate, and complexes with carriers, and other forms. The preferred anti-microbial agent is biquanide.

The composition may also include known plant or seed treatment and fungicidal products such as Vitaflo 280, Apron-Maxx RTA, thiram. The composition may also include seed coatings, enhancers, emulsifiers, thickening agents, solvents, anti foaming agents, preservatives, fragrances, coloring agents, emollients, fillers, and the like.

Multiple inactive ingredients may be optionally incorporated in the formulations. Examples of ingredients are emulsifiers, thickening agents, solvents, anti foaming agents, preservatives, fragrances, coloring agents, emollients, fillers, and the like.

The compositions and methods of the present invention may be used to treat biofilm in a wide range of environments and places. Treating biofilm, as used herein, refers to contacting a biofilm or similar structure with an anti-biofilm agent wherever biofilm may be found, expected to be found, or postulated to be found. One skilled in the art will readily recognize that the areas and industries for which the present invention is applicable is a vast number of processes, products, and places.

The preservative agents incorporated into the matrices and devices of the present invention may be used for a variety of applications where there is a need for the presence of the active agent. A particularly preferred use is in the treatment and preservation of plant materials in both the agricultural and horticultural sectors.

For example, the compositions and methods of the present invention may be effective or beneficial in preserving and/or disinfecting plant seeds. Exemplary seeds include, but are not limited to dry beans, pulse crops (e.g., peas, lentils, chickpeas, and faba beans), seeds from cereals, e.g., corn, wheat and barley; oil seed crops such as canola seeds; seeds of nutraceutical crop plants such as ginseng, and vegetables such as potato seeds.

In this aspect of the invention, the compositions and methods are suitable for treating against one or more microbial infections, including but not limited to diseases or conditions caused by Pseudomonads, Xanthomonads, Curtobacterium species, Sclerotinia. species, Pythium species, Fusarium species Botrytis cinerea, Helminthosporium solani Streptomyces spp, Phytophthora spp., Rhizoctonia solani, Erwinia species, and Clavibacter species, to name just a few.

Exemplary disease or conditions include, but are not limited to bacterial blight, brown spot, common blight, vascular wilt, white mold, root rots, head blight, silver scurf, dry rot, common scab, ring rot, soft rot, damping off, seedling blight, seed rot, and bacterial canker.

The compositions and methods of the present invention are also effective or beneficial in decontaminating, disinfecting, or protecting a wide assortment of surfaces. Exemplary surfaces include, but are not limited to agricultural surfaces, e.g., greenhouses, irrigation systems, storage facilities, and crates and bins; agricultural tools and equipment, including production equipment involved in harvesting, seeding, pruning, tillage and processing/handling equipment such as conveyor belts, pickers, and cutters; food processing plants, centers, or equipment, including dairy plants, poultry plants, slaughter houses, seafood processing plants, fresh produce processing centers, and beverage processing centers.

The compositions and methods of the present invention are also effective or beneficial as a protective coating and/or as an ingredient in a protective coating. Exemplary areas include but are not limited to building, environmental, medical, dental, and industrial surfaces. Exemplary surfaces include but are not limited to hospitals, greenhouses, agricultural storage facilities, water systems, ships (e.g., biocorrosion), cables (e.g., biocorrosion), and pipelines (e.g., biocorrosion); and coatings themselves, e.g., paint, stain, and grout; medical devices, e.g., catheters and dialysis machines, or parts thereof; and dental implants and coatings.

The compositions and methods of the present invention are also effective, or expected to be effective, as a preservative for plant-based cosmetics, including but not limited to an ingredient of a cosmetic, or incorporated into the packaging of a cosmetic.

### DEFINITIONS

As used herein, plant material refers to any plant or vegetable, or parts thereof, including flowers, fruits, produce, seeds, stems, roots, stolons, rhizomes, leaves, vascular system, sprouts, cut flowers, and the like.

One skilled in the art will recognize that a biofilm may be composed of a single species, may be multi-species, homogenous, heterogeneous, and/or may also include other organisms associated with or protected by the biofilm. Biofilm as used herein also refers to one or more stages of biofilm development or formation.

As used herein, anti-biofilm agent refers to any element, chemical, biochemical, or the like that is effective against a biofilm. Typical anti-biofilm agents are those that have anti-microbial, anti-bacterial, anti-fungal or anti-algal properties. Metal and metal compounds, preferably high valency silver ions, have been shown generally to have anti-bacterial and ethylene inhibiting properties, and are preferred anti-biofilm agents in accordance with the present invention. In some embodiments of the invention, the anti-biofilm agent is a broad spectrum agent, e.g., having effectiveness or activity against more than one microbial species.

As used herein, preservatives or similar words include any element, chemical or biochemical or the like that can be used to preserve or extend the shelf like of a plant material, such as a cut flower. A preservative may be an anti-biofilm agent, or may be used in combination with an anti-biofilm agent, or may be used after an anti-biofilm agent is removed or degraded a biofilm.

“Sustained release” or “sustainable basis” are used to define release of atoms, molecules, ions or clusters of a noble metal that continues over time measured in hours or days, and thus distinguishes release of such metal species from the bulk metal, which release such species at a rate and concentration which is too low to be effective, and from highly soluble salts of noble metals such as silver nitrate, which releases silver ions virtually instantly, but not continuously, in contact with an alcohol or electrolyte.

Planktonic: Microorganisms growing as floating, single cells, which is part of their life cycle.

Sustained release: release of atoms, molecules, ions or clusters of an antimicrobial or noble metal that continues over time, measured in hours or days.

Surface contamination, as used herein, refers to microorganisms growing on or relocated to a surface. The microorganisms associated with surface contamination may be actively growing or dormant, but represent a viable inoculum that can reinitiate infection, disease or other undesirable conditions.

Agriculture: includes all sectors, commodities and surfaces associated with plant and food production including but not limited to horticulture, field production, greenhouse production, nursery crops, turf and forages, fiber crops, alternative fuels, and forestry for all phases of production, transport, processing and packaging of plant-derived commodities used for food, fiber, landscaping or recreation. Additionally, agriculture includes all aspects of production, transport, processing and packaging of animal-derived commodities used for food or otherwise. This definition includes any natural or man-made surfaces associated with production, transport, handling, processing and packaging of both plant- and animal-derived commodities.

The present invention will be further described in detail with reference to the following working examples. Note, however, that the present invention is not restricted to these examples.

### EXAMPLES

### Example 1

### Preparation of High Valency Silver Ions

High valency silver ions were prepared using known techniques, as follows: Silver nitrate (Ag(Ag₂O₄)₂NO₃) was prepared through the reaction of aqueous solutions of silver nitrate (AgNO₃) and potassium persulfate (K₂S₂O₈) to yield a black precipitate of pure silver nitrate (see chemical reaction below). The precipitate is recovered by filtration and the powder is dried.

7AgNO₃(aq)+K₂S₂O₈(aq)+8H₂O→Ag(Ag₂O₄)₂NO₃(precipitate)+6HNO₃(aq)+6H₂SO₄(aq)+K₂SO₄(aq)+4H₂(g)

### Description of Starting Materials

| | |
|---|---|
| Silver Nitrate (AgNO₃) | Technical Grade |
| Potassium Persulfate (K₂S₂O₈) | Technical Grade |
| Water | Distilled |

A. clean 1000 L SS Reactor System, equipped with over-head stirrer, charge with deionized water (750 L).
B. Start the agitation and manually charge with 30 kg potassium persulfate (KPS, 110 M).
C. Agitate the mixture until KPS is dissolved.
D. In a clean 250 L vat (plastic or stainless steel) prepare a mixture of de-ionized water (150 L) and silver nitrate (17.85 kg, 105 M). Agitate until dissolved.
E. Maintaining ambient temperature and using a metering pump, transfer the silver nitrate solution to the KPS solution contained in the 1000 L Reactor. A black precipitate will begin to form immediately.
F. Maintain agitation during the addition process (about 30 to 45 minutes).
G. Continue to agitate the reaction mixture for an additional 1 hour. Stop the agitation for 10-20 minutes and siphon off the bulk of the supernatant into a 1500 L vat; hold for later disposal.
H. To the contents of the 1000 L reactor, add de-ionized water (300 L). Agitate the mixture while preparing for filtration.
I. Transfer the aqueous slurry of oxysilvernitrate onto a suitably prepared filter nutsche (pressure/agitated or box) and pull dry. Check the pH of the filtrate.
J. Slurry wash the filter cake with about 15 to 20 L of water and pull dry. Repeat if the filtrate is still acidic (pH<4).
K. Discharge the filter cake to a suitable dryer (tray dryer or agitated pan dryer); determine the weight of the wet material and dry under a stream of air for 12 hours. Determine dry weight and sample for analysis.
L. Transfer the dry product to polyethylene bags and store the product, away from moisture and protected from light.

### Example 2

### High Valency Silver Anti-Microbial Activity Against Erwinia carotovora subsp. carotovora (Ecc), the Soft Rot of Vegetables Pathogen, in Comparison to Nanocrystalline Silver Powder

TABLE 1
Ecc biofilm susceptibility to high valency silver
and nanocrystalline powder Ag30 and Ag 100
(Nanotechnologies, Inc.) at 24 h contact time.

| | Ag30 | Ag100 | Oxy |
|---|---|---|---|
| 500 ppm | 0 | 0 | 0 |
| | 0 | 0 | 0 |
| | 0 | 0 | 0 |
| | 0.00 | 0.00 | 0.00 |
| 200 ppm | 0 | 0 | 0 |
| | 0 | 0 | 0 |
| | 0 | 0 | 2.11 |
| | 0.00 | 0.00 | 0.70 |
| 100 ppm | 0 | 0 | 0 |
| | 0 | 0 | 0 |
| | 0 | 0 | 1.95 |
| | 0.00 | 0.00 | 0.65 |
| 50 ppm | 0 | 1.30 | 1.60 |
| | 1.60 | 0.00 | 1.00 |
| | 1.00 | 1.48 | 2.00 |
| | 0.87 | 0.93 | 1.53 |
| 0 ppm | 3.85 | 3.70 | 3.48 |
| | 3.78 | 3.60 | 3.90 |
| | 3.60 | 3.60 | 3.90 |
| | 3.74 | 3.63 | 3.76 |

Cell counts expressed in log₁₀, silver compound concentration in parts per million.

TABLE 2
Log reduction of Ecc biofilms treated with high
valency silver (Oxy1) and nanocrystalline powder
Ag30 and Ag 100 at 24 h contact time.

| | Ag30 | Ag100 | Oxy1 |
|---|---|---|---|
| 500 ppm | 3.74 | 3.63 | 3.76 |
| 200 ppm | 3.74 | 3.63 | 3.06 |
| 100 ppm | 3.74 | 3.63 | 3.11 |
| 50 ppm | 2.87 | 2.7 | 2.23 |

Conclusion
High valency silver was as efficacious as nanocrystalline silver as an anti-microbial against plant pathogenic Erwinia spp.

### Example 3

### High Valency Silver Anti-Microbial Activity Against Both Biofilm and Planktonics of Pseudomonas syringae pv. phaseolicola HB-9, a Bean Halo Blight Pathogen, in Comparison to Other Seed Treatment Products Such as Copper Sulfate

TABLE 3
Pseudomonas syringae pv. phaseolicola HB-9
biofilm and planktonic susceptibility to high valency
silver and copper based seed treatment products.

| | Bacteria/growth | Bacteria/biofilm growth |
|---|---|---|
| Compound/ | PspHB-9 | PspHB-9 |
| concentration | Planktonic | Biofilm |
| High valency | | |
| silver | | |
| 100 ppm | 0 | 0 |
| 500 ppm | 0 | 0 |
| 1000 ppm | 0 | 0 |
| Copper sulfate | | |
| 1250 ppm | 2.95 | 5.15 |
| 2900 ppm | 0 | 0 |
| 4600 ppm | 0 | 0 |
| H₂0 (0 ppm) | | |
| | 5.66 | 6.05 |
| | 5.75 | 5.97 |
| | 5.75 | 6.67 |
| | 5.72 | 6.23 |

Cell counts expressed in log10, silver compound concentration in parts per million.

TABLE 4
Log reduction of Pseudomonas syringae pv. phaseolicola
(PspHB-9) planktonic and biofilms treated with high
valency silver and copper sulfate for 2 h.

| | Bacteria/growth | Bacteria/biofilm growth |
|---|---|---|
| Compound/ | PspHB-9 | PspHB-9 |
| concentration | Planktonic | Biofilm |
| High valency | | |
| silver | | |
| 100 ppm | 5.72 | 6.23 |
| 500 ppm | 5.72 | 6.23 |
| 1000 ppm | 5.72 | 6.23 |
| Copper sulfate | | |
| 5,000 ppm | 2.77 | 1.08 |
| 11,460 ppm | 5.72 | 6.23 |
| 18,300 ppm | 5.72 | 6.23 |
| H₂0 (0 ppm) | | |
| | 5.66 | 6.05 |
| | 5.75 | 5.97 |
| | 5.75 | 6.67 |
| | 5.72 | 6.23 |

Conclusions
High valency silver led to 100% eradication of planktonic and biofilm at the lowest concentration (50× lower than the lowest copper concentration tested, which did cause reduction in biofilms).
Copper sulfate: ~48% reduction of planktonics and 17% reduction of biofilms at lowest concentration (5000 ppm). A concentration of 11460 ppm was required for it to be effective.
High valency silver is at least >50 times to 114× more effective at eradicating planktonic cells and biofilms PspHB-9 than copper sulfate.

### Example 4

### Evaluation of High Valency Silver on Dry Bean Seed Germination and Emergence—Preliminary Toxicity Analysis Conducted in Greenhouse

Germination was measured directly for 50 seeds from each treatment at 5- and 10-days after treating with the compounds listed in Table 5 and plating on solid agar media. Germination was also measured indirectly for each treatment as emergence of seedlings from 20 seeds sown five/pot in four pots in the greenhouse. The results for germination on plates are shown in Tables 6 and 7. Statistical comparisons of germination data using single variable ANOVA revealed that there were no significant differences in germination of seeds from any of the treatments. These results indicated that the treatments applied to dry bean seeds, including the experimental products, had no significant deleterious effect on seed germination.

TABLE 5
Products and compounds tested

| Product | Formulation | Rate |
|---|---|---|
| Negative Control #1 | Sterile Water | 5-mL or 1-kg per 1-kg seed |
| Negative Control #1 | Talc powder | 5-g to 1-kg seed |
| Ag Streptomycin (positive | 62.6% streptomycin sulphate; 50% | 5 mL of 1% solution to 1 kg seed |
| standard) | streptomycin base | |
| Experimental #2A-wet | Oxysilver nitrate salts in water | 5-mL of 0.05% solution to 1 kg seed |
| Experimental #2B-wet | Oxysilver nitrate salts in water | 5-mL of 0.1% solution to 1 kg seed |
| Experimental #2A- dry | Oxysilver nitrate salts in talc powder | 5-g of 0.05% talc mixture to 1 kg seed |
| Experimental #2B- dry | Oxysilver nitrate salts in talc powder | 5-g of 0.1% talc mixture to 1 kg seed |

TABLE 6
Germination of dry bean (Phaseolus vulgaris L. cv. Othello)
seeds treated with high valency silver on King's B agar plates.

| | | | | High valency | High valency | High valency | High valency |
|---|---|---|---|---|---|---|---|
| | Sterile | Talc | Ag-Strep | silver 0.05% | silver 0.1% | silver 0.05% | silver 0.1% |
| | Water | Powder | 1% | in water | in water | in talc | in talc |
| Plate 1 | 5 | 4 | 5 | 5 | 5 | 5 | 4 |
| Plate 2 | 5 | 5 | 4 | 5 | 5 | 5 | 5 |
| Plate 3 | 5 | 5 | 5 | 5 | 5 | 5 | 5 |
| Plate 4 | 5 | 5 | 5 | 5 | 5 | 5 | 5 |
| Plate 5 | 5 | 5 | 5 | 5 | 5 | 5 | 4 |
| Plate 6 | 4 | 5 | 5 | 5 | 5 | 5 | 5 |
| Plate 7 | 5 | 5 | 4 | 5 | 5 | 5 | 5 |
| Plate 8 | 5 | 5 | 5 | 5 | 5 | 5 | 5 |
| Plate 9 | 5 | 5 | 4 | 5 | 5 | 5 | 5 |
| Plate 10 | 5 | 5 | 4 | 5 | 5 | 5 | 5 |
| SUM | 49 | 49 | 46 | 50 | 50 | 50 | 48 |
| MEAN | 4.9 | 4.9 | 4.6 | 5 | 5 | 5 | 4.8 |

TABLE 7
Germination of dry bean (Phaseolus vulgaris L. cv. AC Polaris) seeds
treated with high valency silver nitrate on King's B agar plates.

| | | | | High valency | High valency | High valency | High valency |
|---|---|---|---|---|---|---|---|
| | Sterile | Talc | Ag-Strep | silver 0.05% | silver 0.1% | silver 0.05% | silver 0.1% |
| | Water | Powder | 1% | in water | in water | in talc | in talc |
| Plate 1 | 5 | 3 | 5 | 5 | 4 | 5 | 5 |
| Plate 2 | 4 | 3 | 4 | 3 | 5 | 5 | 5 |
| Plate 3 | 4 | 4 | 3 | 5 | 3 | 5 | 5 |
| Plate 4 | 4 | 4 | 3 | 4 | 1 | 4 | 5 |
| Plate 5 | 4 | 5 | 5 | 2 | 4 | 5 | 4 |
| Plate 6 | 4 | 4 | 4 | 3 | 4 | 5 | 4 |
| Plate 7 | 3 | 3 | 4 | 4 | 3 | 5 | 5 |
| Plate 8 | 3 | 3 | 4 | 5 | 5 | 5 | 4 |
| Plate 9 | 4 | 3 | 3 | 4 | 4 | 4 | 5 |
| Plate 10 | 5 | 3 | 3 | 3 | 5 | 3 | 4 |
| SUM | 40 | 35 | 38 | 38 | 38 | 46 | 46 |
| MEAN | 4 | 3.5 | 3.8 | 3.8 | 3.8 | 4.6 | 4.6 |

### Example 5

### Emergence of Seedlings in the Greenhouse

TABLE 8
Emergence of dry bean (Phaseolus vulgaris L. cv. Othello) seedlings from seeds treated
with high valency silver in preliminary greenhouse trials, three weeks after planting.

| | | | | High valency | High valency | High valency | High valency |
|---|---|---|---|---|---|---|---|
| | Sterile | Talc | Ag-Strep | silver 0.05% | silver 0.1% | silver 0.05% | silver 0.1% |
| Replication | Water | Powder | 1% | in water | in water | in talc | in talc |

Experiment 1 - Emergence Data For ‘Othello’ Greenhouse Trial #1

| | | | | | | | |
|---|---|---|---|---|---|---|---|
| 1 | 3 | 4 | 5 | 5 | 4 | 4 | 4 |
| 2 | 3 | 5 | 3 | 4 | 4 | 4 | 3 |
| 3 | 5 | 4 | 3 | 5 | 4 | 5 | 5 |
| 4 | 5 | 5 | 3 | 5 | 3 | 5 | 4 |
| SUM | 16 | 18 | 14 | 19 | 15 | 18 | 16 |
| MEAN | 4 | 4.5 | 3.5 | 4.75 | 3.75 | 4.5 | 4 |

Experiment 2 - Emergence Data For ‘Othello’ Greenhouse Trial #2

| | | | | | | | |
|---|---|---|---|---|---|---|---|
| 1 | 4 | 4 | 5 | 5 | 4 | 5 | 4 |
| 2 | 5 | 3 | 4 | 5 | 5 | 4 | 4 |
| 3 | 5 | 4 | 5 | 5 | 5 | 5 | 5 |
| 4 | 4 | 3 | 5 | 5 | 5 | 5 | 4 |
| SUM | 18 | 14 | 19 | 20 | 19 | 19 | 17 |
| MEAN | 4.5 | 3.5 | 4.75 | 5 | 4.75 | 4.75 | 4.25 |

Total and Mean of Trials 1 and 2 for ‘Othello’ seedling emergence

| | | | | | | | |
|---|---|---|---|---|---|---|---|
| TOTAL | 34 | 32 | 33 | 39 | 34 | 37 | 33 |
| MEAN | 4.25 | 4 | 4.125 | 4.875 | 4.25 | 4.625 | 4.125 |

TABLE 9
Emergence of ‘AC Polaris’ seedlings of treated with high valency
silver in preliminary greenhouse trials three weeks after planting.
Experiment 3 - Emergence Data For ‘AC Polaris’ Greenhouse Trial #1

| | | | | High valency | High valency | High valency | High valency |
|---|---|---|---|---|---|---|---|
| | Sterile | Talc | Ag-Strep | silver 0.05% | silver 0.1% | silver 0.05% | silver 0.1% |
| Replication | Water | Powder | 1% | in wwater | in water | in talc | in talc |
| 1 | 5 | 4 | 5 | 4 | 3 | 4 | 5 |
| 2 | 4 | 5 | 2 | 4 | 4 | 4 | 5 |
| 3 | 3 | 5 | 4 | 3 | 4 | 5 | 5 |
| 4 | 2 | 5 | 2 | 4 | 5 | 5 | 5 |
| SUM | 14 | 19 | 13 | 15 | 16 | 18 | 20 |
| MEAN | 3.5 | 4.75 | 3.25 | 3.75 | 4 | 4.5 | 5 |

### Example 5

### Microbial Recovery from Dry Bean (Phaseolus vulgaris L. cvs. Othello and AC Polaris) Seeds Treated with High Valency Silver in Comparison to Untreated Seeds

Dry Bean Seed Cultivars Used in these Experiments: Sample Description
1. ‘Othello’—Pinto beans—These seeds were collected straight from the field harvester prior to sorting or cleaning. They included seeds that would normally be discarded during processing in a commercial seed treatment facility—damaged, discoloured, shrivelled or small seeds. Due to the fact they were not cleaned these seeds still contain soil, dirt, plant debris, etc. Therefore, this seed lot was expected to provide a good model for a high natural microbial challenge, with good diversity of organisms and high microbial recovery expected. These seeds were overall more challenging to treat, generating more variable results as expected for such minimally processed seeds.
2. AC Polaris—White beans—These seeds were processed and cleaned in a commercial seed treatment facility. Processing leads to rejection of small, damaged, and/or discoloured seeds, and thus a healthier overall seed lot. After processing, seeds were washed and cleaned, resulting in a significant reduction in soil, dirt or debris. Due to these processing steps, lower diversity and numbers of microorganisms were expected to be recovered from these seeds, especially in comparison to the highly challenging lot of ‘Othello’ seeds used in this study. The ‘AC Polaris’ seed lot was an excellent source for testing of artificially inoculated seeds.

### Scoring of Seed Microbial Contamination

Experiments were conducted by treating lots of ‘Othello’ and ‘AC Polaris’ seeds with challenges described in Table 10. Seeds were placed individually onto wells of 12-well microtiter plates and treated for 1 h with silver and/or water. After treatment, seeds were rinsed by dipping into fresh phosphate buffer and subsequently transferred to a new 12-well microtiter plate containing fresh phosphate buffer and sonicated for 15-30 min. Sonication allowed for surviving bacterial and fungal to be removed from the seed surface and recovered into the liquid. After 6 days, each of the wells containing seeds were scored based on both:
the turbidity of the liquid in which they were originally immersed for treatment, on day 6.
seed surface area covered by bacterial and/or fungal colonization, also on day 6.

Scores varied from:
(0)—No microorganisms observed on seed surfaces and no turbidity evident after 6 days
(1)—Minimal turbidity and/or no more than 20% of the external area of the seed visibly covered by microorganisms
(2)—More than 20% but less than 50% of seed surface covered by microorganisms and/or turbidity above that observed on (1) but far from matching a McFarland turbidity standard scale of 0.5.
(3)—At least 50% of seed surface colonized by microorganisms and heavier turbidity approaching the turbidity in the lowest standard of McFarland scale—0.5.
(4)—More than 50% of seed surface colonized by bacteria and/or fungi and/or heavy turbidity (bottom of well of 12-well plate barely visible)
(5)—Seed surface completely covered by microorganisms and/or maximum turbidity, “milky” looking liquid cultures.

TABLE 10
Description of experiments.

| Experiment | Objective |
|---|---|
| 1. Microbial recovery from non-inoculated, | To determine the type and number of naturally |
| non-sterile seeds. | occurring microorganisms that could be removed |
| | from the bean seed coat and cultured on agar |
| | medium. |
| 2. Microbial recovery from non-sterilized, | To determine the efficacy of high valency silver |
| non-inoculated seeds treated with high | as a bean seed treatment, by measuring |
| valency silver. | eradication of naturally occurring microorganisms |
| | from the seed surface. |
| 3. Microbial recovery from non-sterilized | Same as Experiment #1 except that seeds were |
| seeds inoculated with Pseudomonas | artificially infested with the halo blight pathogen, |
| syringae pv. phaseolicola. | Pseudomonas syringae pv. phaseolicola. |
| 4. Microbial recovery from non-sterilized | Same as Experiment #2 except that seeds are |
| seeds inoculated with Pseudomonas | artificially infested with the halo blight pathogen, |
| syringae pv. phaseolicola treated with high | Pseudomonas syringae pv. phaseolicola.. |
| valency silver. | |

Six seeds were scored in each experiment and each experiment was repeated 3 times independently. Numbers presented on Tables 11, 13, and 15 represent the mean of 6 scores. Efficacy of high valency silver in reducing the microbial populations on seeds was measured based on overall scores for bacteria, fungi. Total microbial scores (bacteria and fungi together) and are represented as percentage of killing on Tables 12, 14, and 16.

### Experiment 1

‘Othello’ seeds: Fungi with four distinct colony morphologies were recovered from ‘Othello’ seeds, mostly on non-inoculated treatments. Visual identifications included those with spore coloration indicative of Fusarium sp. Five to six types of bacterial colonies were also recovered from non-inoculated ‘Othello’ seeds. When seeds were inoculated with the halo blight pathogen, fungal occurrence and numbers were reduced, which is not surprising due to the broad spectrum of antibiotics produced by bacteria from the genus Pseudomonas in general. These antibiotics can provide a significant competitive advantage versus other microorganisms when colonizing surfaces.

‘AC Polaris’ seeds: ‘AC Polaris’ seeds had been pre-cleaned in a commercial seed cleaner facility, and recovery data confirmed the previously mentioned expectation of lower microbial recovery from these seeds when non-inoculated. Interestingly, when these seeds were inoculated with P. syringae pv. phaseolicola bacterial counts were unusually high on untreated seeds. This may be due to the reduction in competitive advantage and succession of species on the surface when no additional stresses were applied.

Data are summarized in tables 11 and 12 below.

TABLE 11
Estimation of bacterial and fungal populations recovered from untreated
‘Othello’ and ‘AC-Polaris’ seed surfaces in comparison to high valency
silver-treated seed surfaces, based on a scoring system.

| | | | | Total microbial |
|---|---|---|---|---|
| Seed variety | Non-inoculated | Bacteria | Fungi | score |
| Othello | H₂0 | 4.0 | 3.83 | 7.83 |
| Othello | High valency silver | 0.83 | 1.16 | 1.99 |
| | (0.1%) | | | |
| AC Polaris | H₂0 | 2.0 | 1.5 | 3.5 |
| AC Polaris | High valency silver | 0.66 | 0 | 0.66 |
| | (0.1%) | | | |
| | Inoculated w/ | | | Total microbial |
| Seed variety | bacteria | Bacteria | Fungi | score |
| Othello | H₂0 | 3.66 | 1.5 | 5.16 |
| Othello | High valency silver | 1.5 | 0.33 | 1.83 |
| | (0.1%) | | | |
| AC Polaris | H₂0 | 5.0 | 0 | 5 |
| AC Polaris | High valency silver | 0 | 0 | 0 |
| | (0.1%) | | | |

TABLE 12
Efficacy of high valency silver compound in reducing microbial
infestation of seeds, showing estimated reduction of bacterial and
fungal population, in addition to overall microbial load (bacteria
and fungi) as a percentage of that observed on untreated seeds.

| High Valency Silver | Bacterial | Fungal | Total microbial |
|---|---|---|---|
| Treatment | reduction | reduction | reduction |
| Othello Non-inoculated | 80% | 70% | 75% |
| AC Polaris Non-inoculated | 67% | 100% | 81% |
| Othello Inoculated | 41% | 78% | 65% |
| AC Polaris Inoculated | 100% | Non applicable | 100% |

### Experiment 2

The same trend observed in Experiment 1 was observed in experiment 2.

‘Othello’ seeds: Fungi with three distinct colony morphologies were recovered from ‘Othello’ seeds, mostly on non-inoculated treatments, based on preliminary visual identification. Five types of bacterial colonies were recovered from non-inoculated ‘Othello’ seeds. When seeds were inoculated with the halo blight pathogen, fungal occurrence was reduced, and organisms recovered were mainly Pseudomonas.

‘AC Polaris’ seeds: Recovery data showed lower microbial recovery from these seeds on natural, non-inoculated seeds. When inoculated with the halo blight pathogen, bacterial counts were high on untreated seeds.

Data is summarized in Tables 13 and 14.

TABLE 13
Estimation of bacterial and fungal populations recovered from
untreated ‘Othello’ and ‘AC-Polaris’ seed surfaces in comparison to
high valency silver-treated seed surfaces, based on a scoring system.

| | | | | Total microbial |
|---|---|---|---|---|
| Seed variety | Non-inoculated | Bacteria | Fungi | score |
| Othello | H₂0 | 2.16 | 3.66 | 5.82 |
| Othello | High valency silver | 0.83 | 0 | 0.83 |
| | (0.1%) | | | |
| AC Polaris | H₂0 | 1.66 | 1.33 | 2.99 |
| AC Polaris | High valency silver | 0.16 | 0.5 | 0.66 |
| | (0.1%) | | | |
| | Inoculated w/ | | | Total microbial |
| Seed variety | bacteria | Bacteria | Fungi | score |
| Othello | H₂0 | 3.00 | 2.66 | 5.66 |
| Othello | High valency silver | 0.66 | 0.66 | 1.32 |
| | (0.1%) | | | |
| AC Polaris | H₂0 | 4.16 | 0 | 4.16 |
| AC Polaris | High valency silver | 0.16 | 0 | 0.16 |
| | nitrate 1 (0.1%) | | | |

TABLE 14
Efficacy of high valency silver compound in reducing microbial
infestation of seeds, showing estimated reduction of bacterial and
fungal population, in addition to overall microbial load (bacteria
and fungi) as a percentage of that observed on untreated seeds.

| High Valency Silver | Bacterial | Fungal | Total microbial |
|---|---|---|---|
| Treatment | reduction | reduction | reduction |
| Othello Non-inoculated | 62% | 100% | 86% |
| AC Polaris Non-inoculated | 90% | 62% | 78% |
| Othello Inoculated | 78% | 75% | 77% |
| AC Polaris Inoculated | 96% | Non applicable | 96% |

### Experiment 3

Diversity data was similar to previous experiments.

TABLE 15
Estimation of bacterial and fungal populations recovered from untreated
Othello and AC-Polaris seed surfaces in comparison to high valency
silver-treated seed surfaces, based on a scoring system.

| | | | | Total microbial |
|---|---|---|---|---|
| Seed variety | Non-inoculated | Bacteria | Fungi | score |
| Othello | H₂0 | 2.83 | 3.16 | 5.99 |
| Othello | High valency silver | 0.33 | 0.83 | 1.16 |
| | (0.1%) | | | |
| AC Polaris | H₂0 | 2.0 | 1.16 | 3.16 |
| AC Polaris | High valency silver | 0 | 0 | 0 |
| | (0.1%) | | | |
| Othello | H₂0 | 4.16 | 3.66 | 7.82 |
| Othello | High valency silver | 1.66 | 0 | 1.66 |
| | (0.1%) | | | |
| AC Polaris | H₂0 | 4.16 | 0 | 4.16 |
| AC Polaris | High valency silver | 0 | 0 | 0 |
| | (0.1%) | | | |

TABLE 16
Efficacy of high valency silver compound in reducing microbial
infestation of seeds, showing estimated reduction of bacterial and
fungal population, in addition to overall microbial load (bacteria
and fungi) as a percentage of that observed on untreated seeds.

| High Valency Silver | Bacterial | Fungal | Total microbial |
|---|---|---|---|
| Treatment | reduction | reduction | reduction |
| Othello Non-inoculated | 88% | 74% | 81% |
| AC Polaris Non-inoculated | 100% | 100% | 100% |
| Othello Inoculated | 60% | 100% | 79% |
| AC Polaris Inoculated | 100% | Non applicable | 100% |

Conclusions:
High valency silver showed good bactericidal and fungicidal efficacy against various bacterial and fungal species associated with seed surfaces. The data was consistent through 3 independent replications of this experiment.

### Example 6

### Evaluating of the Effects of High Valency Silver when Applied as an Aqueous Coating to Seeds of Various Dry Bean Cultivars

The efficacy of high valency silver ions produced by oxysilver nitrate was evaluated as a seed treatment on diseased dry bean seeds. The oxysilver nitrate was applied as an aqueous seed coating on the germination of dry bean (Phaseolus vulgaris L).

High valency silver ions were prepared using known techniques, as follows: Silver nitrate (Ag(Ag₂O₄)₂NO₃) was prepared through the reaction of aqueous solutions of silver nitrate (AgNO₃) and potassium persulfate (K₂S₂O₈) to yield a black precipitate of pure silver nitrate (see chemical reaction below). The precipitate is recovered by filtration and the powder is dried.

7AgNO₃(aq)+K₂S₂O₈(aq)+8H₂O→Ag(Ag₂O₄)₂NO₃(precipitate)+6HNO₃(aq)+6H₂SO₄(aq)+K₂SO₄(aq)+4H₂(g)

Seeds were placed on blotters in Petri plates, were soaked in an excess of water for 3 days, then drained on day 4. The 3-day period of excess water created a very high bacterial load on the germinating seeds and high disease pressure due to pathogenic, saprophytic and soft-rot bacteria. This disease pressure was so extreme that the germination rates for experiments 2-5 were essentially zero due to being overwhelmed by the microbial growth. However experiment #1 (a healthy seed lot of cultivar ‘Othello’) was able to overcome the disease pressure and to germinate significantly.

These extreme conditions were used to uncover the pronounced effectiveness of high valency silver against bacterial disease pressure. Under these conditions, high valency silver treatment significantly increased germination compared with a negative control (water) and a positive standard (Apron Maxx RTA).

By day 10, the 1% high valency silver treatment increased germination by 34.7% compared with the negative control (sterile water). In addition, germination was 54% higher than Apron Maxx RTA—a registered seed treatment and industry standard for dry bean. The results are shown in Table 1 and graphically in FIG. 1. The treatments with high valency silver had no negative effects on germination as seen in the number of days needed to reach 25% germination. For example, phytotoxic compounds applied to seeds will delay or prevent germination. This will cause a longer time period required to reach 25% and/or 50% germination.

Furthermore, high valency silver increased the number of seeds germinated by 1.3- to 2.3-times. The positive effects of high valency silver can be visually observed in FIG. 2.

High valency silver is an excellent dry bean seed treatment because it has no phytotoxic effect on germinating seeds and can significantly rescue germination in seeds challenged with high numbers of contaminating bacteria. Specifically, it demonstrates that germination rates can be increased using high valency silver as a seed treatment, especially in lower quality seeds or poor germination conditions. It was also noted for other pulse crops that high valency silver treatment could accelerate germination (see Example 7), however no treatment in the experiment described herein led to accelerated germination of dry bean seeds.

Results of this experiment are shown in the following Table 17.

TABLE 17
Germination results for Experiment 6.

| | # seeds | | Day to 25% |
|---|---|---|---|
| Treatment | germinated | % germination | germination |
| 0.1% high valency silver | 53 | 35.3 | 5 |
| 0.25% high valency silver | 51 | 43.0 | 5 |
| 0.5% high valency silver | 53 | 35.3 | 5 |
| 1% high valency silver | 91 | 60.7 | 5 |
| Sterile water | 39 | 26.0 | 5 |
| Apron Maxx RTA | 10 | 6.7 | n/a |

### Example 7

### Evaluation of Phytotoxicity of High Valency Silver on Pulse Crop Seeds (Pea Chickpea, Lentil, Soybean, Dry Bean)

This experiment determines whether the same high valency silver as that used in Example 6, when added to pulse seeds as an antimicrobial seed treatment, reduces germination, emergence or is phytotoxic to the pulse crops pea, chickpea, soybean and lentil. Germination data were recorded for seeds to which high valency silver was applied as an aqueous coating at one of four concentrations: 1000-ppm, 2500-ppm, 5000-ppm and 10000-ppm.

One hundred seeds each of five pulse crops (Table 18) were treated with the various concentrations of high valency silver (Table 19) and then sown (n=50 seeds) in non-sterile sandy soil in 5″ pots and placed in a greenhouse, or placed on a moist blotter (n=50 seeds) in sterile Petri plates. Germination on blotters was scored at 7- and 14-days. Emergence from soil was scored at 21- and 28-days. Germination and emergence of pulse seeds coated with high valency silver were compared with results for seeds treated with water, and seedlings were visually rated for any signs of phytotoxicity. Controlled variables for this experiment are summarized in Table 20.

To treat seeds, one hundred seeds were placed in a 50-mL Falcon tube and combined with the treatment solution. Seeds were mixed with the treatment for 5-min by gently rolling and inverting the Falcon tube. Coated seeds were placed in an open Petri plate for 30-min to dry. Seeds were sown 10-per pot, set in a greenhouse, and maintained under standard conditions with mercury lamp lighting supplementing daylight from 3:00 pm to 10:00 pm.

TABLE 18
Experimental factors for pulse germination and emergence trials.

| | |
|---|---|
| Experimental Factors | Treatments |

Pulse Seeds

| | |
|---|---|
| A1 | Camry (Green Pea) |
| A2 | Myles (Dezi Chickpea) |
| A3 | Frontier (Kabuli Chickpea) |
| A4 | Plato (Large Green Lentil) |
| A5 | Orford (Soybean) |

Treatments and Checks

| | |
|---|---|
| B1 | 0.1% high valency silver |
| B2 | 0.25% high valency silver |
| B3 | 0.5% high valency silver |
| B4 | 1% high valency silver |
| B5 | Water |

TABLE 19
Treatment solutions and concentrations.

| Treatment | Concentration (ppm) | Concentration (%) |
|---|---|---|
| high valency silver | 1000 | 0.1% |
| high valency silver | 2500 | 0.25% |
| high valency silver | 5000 | 0.5% |
| high valency silver | 10000 | 1% |
| Sterilized water | N/A | N/A |

TABLE 20
Experimental factors for pulse germination and emergence trials.

| | |
|---|---|
| Experimental Factors | Treatments |

Pulse Seeds

| | |
|---|---|
| A1 | Camry (Green Pea) |
| A2 | Myles (Dezi Chickpea) |
| A3 | Frontier (Kabuli Chickpea) |
| A4 | Plato (Large Green Lentil) |
| A5 | Orford (Soybean) |

Treatments and Checks

| | |
|---|---|
| B1 | 0.1% high valency silver |
| B2 | 0.25% high valency silver |
| B3 | 0.5% high valency silver |
| B4 | 1% high valency silver |
| B5 | Water |

### Preparation of Treatment Solutions:

10% solution—add 0.5-g of high valency silver to 5-mL of sterile water. Stir constantly.
5% solution—after the 10% solution has equilibrated for 15-min, add 0.5-mL of 10% solution to 0.5-mL of sterile water. Stir constantly.
2.5% solution—add 0.25-mL of 10% solution to 0.75-mL of sterile water. Stir constantly.
1% solution—add 0.1-mL of 10% solution to 0.9-mL of sterile water. Stir constantly.

### Treatment of Pulse Seeds With High Valency Silver:

Place 100-seeds into a 50-mL Falcon tube and add 1-mL of treatment solution. Gently roll and invert the tube for 5 min to evenly coat each seed without causing damage. Remove seeds from tube by pouring carefully into an empty Petri plate. Leave exposed (lid off) and air dry seeds for 30 min.

### Assessment Of Germination and Emergence

(1) Germination: Place 50 seeds from each treatment onto 25 individual moistened blotters in large Petri plates. Incubate at room temperature for 2-weeks with or without light. Score germination at 7- and 14-days.
(2) Emergence: Sow 50 seeds from each treatment into five pots (10-seeds per pot and five pots per treatment). Place in a greenhouse at 22° C. with ample air circulation. Use mercury lamps to supplement lighting (on at 3:00 pm; off at 10:00 pm) if necessary. For pre-emergent plants, water pots as needed to keep soil moist but not wet. Water emergent plants daily. Score emergence at 21 and 28 days.

Seeds were treated with water, or one of four concentrations of high valency silver and air dried in Petri plates. After seeds were treated and air-dried, they were sown in potted, non-sterile soil in a greenhouse. Alternatively they were placed on moist blotters in large Petri plates.

Germination of seeds on blotters was scored at 7-days (Table 21) and 14-days (Table 22). All soybean seeds had very slow and low rates for germination with many seeds un-germinated after 7-days and a maximum germination rate of 64%. Kabuli chickpea also had overall reduced germination rates for all treatments, including the control, although the reduction in germination for Kabuli chickpea was less severe than that for soybean.

The treatments with high valency silver had no negative effects on germination of any pulse seed in this experiment. On the contrary, seeds with heavy microbial loads (like soybean) showed increasing rates of germination directly related to increasing concentrations of high valency silver used to treat the seeds.

TABLE 21
Germination of pulse seeds.

| | | 0.1% high | 0.25% high | 0.5% high | 1% high |
|---|---|---|---|---|---|
| | | valency | valency | valency | valency |
| | Water | silver | silver | silver | silver |
| Chickpea | 45 | 47 | 45 | 44 | 45 |
| ‘Myles’ | | | | | |
| Chickpea | 42 | 40 | 45 | 48 | 47 |
| ‘Frontier’ | | | | | |
| Lentil | 48 | 46 | 46 | 49 | 42 |
| Pea | 43 | 46 | 48 | 48 | 47 |
| Soybean | 4 | 7 | 8 | 10 | 19 |

Scores are out of 50 seeds that had been on blotters for 7-days.

TABLE 22
Germination of pulse seeds.

| | | 0.1% high | 0.25% high | 0.5% high | 1% high |
|---|---|---|---|---|---|
| | | valency | valency | valency | valency |
| | Water | silver | silver | silver | silver |
| Chickpea | 44 | 44 | 46 | 47 | 45 |
| ‘Myles’ | | | | | |
| Chickpea | 34* | 45 | 41 | 48 | 45 |
| ‘Frontier’ | | | | | |
| Lentil | 48 | 49 | 48 | 49 | 47 |
| Pea | 47 | 45 | 46 | 48 | 46 |
| Soybean | 15 | 25 | 27 | 29 | 32 |

Scores are out of 50 seeds that had been on blotters for 14-days.
*Germination rates are reduced at 14-days (when compared to data at 7-days in Table 22) because some emerging hypocotyls were observed at 7-days and scored positive for germination. However, subsequent development was arrested and the hypocotyls never fully emerged. Therefore at 14-days these seeds were scored as a negative for germination.

### Example 8

### Enhancement of Germination by High Valency Silver

This experiment evaluates the enhancement of germination by high valency silver when applied to a lower quality soybean seed.

High valency silver was found to increase the speed and number of germinated soybean seeds. The speed of germination is seen in the percent germination after 7-days (Table 23). The water control reaches 26.7% by day seven but the high valency silver treatments reach 28%, 29.6%, 34.5%, and 59.4% respectively.

An increase in number of germinated seeds treated with high valency silver is seen when compared with water alone. After 14 days, the 1% high valency silver has a germination rate 34% higher than water (Table 24). The high valency silver treatments effectively double (or nearly double) percent germinated seeds.

Excessive bacterial growth was seen on the seeds and blotters from the water treated seeds while the high valency silver treated seeds did not appear to have major bacterial growth.

TABLE 23
Germination of pulse seeds.

| | | 0.1% high | 0.25% high | 0.5% high | 1% high |
|---|---|---|---|---|---|
| | | valency | valency | valency | valency |
| Day 7 | Water | silver | silver | silver | silver |
| Soybean | 4 | 7 | 8 | 10 | 19 |
| % of seeds | 26.7 | 28 | 29.6 | 34.5 | 59.4 |
| germinated | | | | | |

Scores are out of 50 seeds that had been on blotters for 7-days.

TABLE 9
Germination of pulse seeds.

| | | 0.1% high | 0.25% high | 0.5% high | 1% high |
|---|---|---|---|---|---|
| | | valency | valency | valency | valency |
| Day 14 | Water | silver | silver | silver | silver |
| Soybean | 15 | 25 | 27 | 29 | 32 |
| % germination | 30 | 50 | 54 | 58 | 64 |

Scores are out of 50 seeds that had been on blotters for 14-days.

### Example 9

### Healthy Dry Bean Seed

Healthy dry bean seed treated with high valency silver had more rapid germination and higher percent germination rate (Table 25) than water control or the industry standard ‘Apron Maxx RTA’. Additionally, the shortest time to 50% and 100% germination was seen in the high valency silver treatments (Table 25).

TABLE 25
Germination results for healthy dry bean seed.

| | | % | | |
|---|---|---|---|---|
| | # seeds | germi- | Day to 50% | Day to 100% |
| Treatment | germinated | nation | germination | germination |
| 0.1% high valency | 84 | 84 | 6.5 | 9.0 |
| silver | | | | |
| 0.25% high valency | 81 | 81 | 5.0 | 9.0 |
| silver | | | | |
| 0.5% high valency | 86 | 86 | 6.0 | 9.0 |
| silver | | | | |
| 1% high valency | 89 | 89 | 5.0 | 9.0 |
| silver | | | | |
| Sterile water | 82 | 82 | 6.0 | 10.0 |
| Apron Maxx RTA | 63 | 63 | 5.5 | 10.0 |

We claim:

1. A method for treating plant material comprising contacting a plant material or a portion thereof with a composition that comprises at least one preservative agent, said preservative agent comprising a high valency silver ion.

2. The method claim 1 wherein said composition comprises:
an aqueous solution of silver nitrate;
an oxidizing agent; and
anions of at least one acid

3. The method of claim 1 wherein contacting a plant or portion thereof includes contacting a seed, root, rhizome, stolon, tuber, stem, leaf, flower, vascular tissue, and combinations thereof.

4. The method of claim 1 wherein preserving the plant material comprises treating the plant material against one or more biofilms.

5. The method of claim 4 wherein treating the plant material against one or more biofilms comprises eradicating or reducing the biofilm.

6. The method of claim 4 wherein treating the plant material against one or more biofilms comprises treating the plant material against one or more species selected from the group consisting of Erwinia species, Pseudomonas species, Xanthomonas species, Clavibacter species, Curtobacterium species, Streptomyces species, Fusarium species, Rhizoctonia species, Colletotrichum species, Verticillium species, Pythium species, Phythophthora species, Helminthosporium species, Sclerotinia species, Botrytis species, Ascochyta species, and variants thereof.

7. (canceled)

8. (canceled)

9. The method of claim 4 wherein the biofilm is located on or in a plant or portion thereof, or a natural or man-made surface material associated with the production, transport, handling, processing or packaging of a plant commodity.

10. (canceled)

11. (canceled)

12. The method of claim 4 wherein the biofilm is located on or in a industrial machine or surface or portion thereof.

13. The method of claim 4 further comprising at least one additional anti-biofilm agent.

14. The method of claim 4 wherein said anti-biofilm agent further comprises treating planktonic bacteria.

15. A composition for treating plants comprising a composition comprising high valency silver ions.

16. (canceled)

17. (canceled)
